(19) United States
(12) Patent Application Publication
(10) Pub. No.: US 2019/0358072 A1
(43) Pub. Date: Nov. 28, 2019

(54) KNEE BRACE

(71) Applicant: INDALO STUDIO INC., Montreal (CA)

(72) Inventor: DANIEL LAROUCHE, MONTREAL (CA)

(21) Appl. No.: 15/988,391

(22) Filed: May 24, 2018

(51) Int. Cl. A61F 5/01

(52) U.S. Cl. CPC A61F 5/0123; A61F 2005/0158; A61F 2005/0167

(57) ABSTRACT

A knee brace for supporting and stabilizing a knee. The knee brace includes an upper frame member, a lower frame member, a plurality of pivoting brackets, each pivoting bracket pivotably linking the upper frame member to the lower frame member, at least one retention strap, each retention strap holding the knee brace on the knee, a tightening mechanism for controlling the rigidity of the knee brace and the circular compression around the knee, and a receptacle for receiving the tightening mechanism. The receptacle is integrated directly into the lower frame member.

### FIELD OF THE INVENTION

The present invention relates to a knee brace, and more specifically to a knee brace for supporting and stabilizing a knee.

### BACKGROUND OF THE INVENTION

Orthoses are externally applied devices used to modify the structural and functional characteristics of the neuromuscular and skeletal system. A common type of orthosis is a knee orthosis or knee brace, which is worn by an individual and extends above and below the knee joint to support or align the knee. Knee braces may be worn to prevent injury or to treat such injuries as bursitis, tendonitis, torn ligaments, worn-out cartilages, arthritis, muscle sprains, and muscle strains. They are typically made from combinations of metal, foam, plastic, and elastic materials and straps.

Hinged or rigid knee braces typically comprise various tightening mechanisms such as straps to secure the brace to the user's leg. However, the tightening mechanisms in common knee braces only tighten the brace around the leg rather than increase the brace's overall rigidity. In addition, in current knee braces that utilize such tightening mechanisms, there must be a retention means such as an external receptacle to connect the tightening mechanism to the knee brace, which requires extra components that complicate the brace, increase costs and increase the risk of damage or failure.

Finally, current rigid knee braces typically comprise an upper section and a lower section that are joined by a pivoting bracket to allow for the necessary articulation of the knee, with bushings that are inserted into the pivoting bracket. These bushings tend to break often, necessitating their replacement to restore the knee brace to properly functioning order.

### SUMMARY OF THE INVENTION

It is therefore a general object of the present invention to provide an improved knee brace with a tightening mechanism that increases the rigidity of the brace and offers circular compression of the brace around the knee.

It is a further object of the present invention to provide an improved knee brace with a receptacle for a tightening mechanism that is built directly into the brace.

It is a further object of the present invention to provide an improved knee brace with a reliable and solid pivoting bracket.

In order to address the above and other drawbacks, there is provided a knee brace for supporting and stabilizing a knee, the knee brace comprising an upper frame member, a lower frame member, a plurality of pivoting brackets, each pivoting bracket pivotably linking the upper frame member to the lower frame member, at least one retention strap, each retention strap holding the knee brace on the knee, a tightening mechanism, the tightening mechanism controlling the rigidity of the knee brace and the circular compression around the knee, and a receptacle for receiving the tightening mechanism, the receptacle integrated directly into the lower frame member.

Preferably, the upper frame member, lower frame member and the pivoting brackets are 3D-printed.

In an embodiment, the upper frame member comprises a pair of upper strut members and an upper cross member, and the lower frame member comprises a pair of lower strut members and a lower cross member, wherein each upper strut member is joined to a corresponding lower strut member at a respective the pivoting bracket, the upper cross member applying pressure above the knee, and the lower cross member applying pressure below the knee.

In an embodiment, the receptacle is 3D-printed onto a first one of the lower strut members and comprises a base, a rear receptacle wall and a front receptacle wall, the tightening mechanism insertable between the rear receptacle wall and the front receptacle wall.

In an embodiment, the tightening mechanism comprises a tightener insertable into the receptacle, the tightener comprising a rotatable and telescopable dial, a connector insertable into the tightener to secure the tightening mechanism into place, a tether tethered to the tightener and the connector, the tether circumferentially wrapping around the knee brace, a front pad and a rear pad, each pad applying pressure below the front and the rear of the knee respectively, the tether passing through the front pad and the rear pad, and tether guides attached to a second one of the lower strut members, the tether guides retaining and guiding the tether, wherein the tightening mechanism is tightenable and loosenable by rotating the dial on the tightener, and wherein the tightening mechanism is movable between a locked position and an unlocked position by axially adjusting the dial on the tightener.

In an embodiment, the receptacle further comprises a slot in the base and a plurality of cut-outs in the rear receptacle wall, the slot and plurality of cut-outs engageable with the tightener, the receptacle further comprising a slot in the rear receptacle wall such that the connector is insertable through the slot in the rear connector wall to connect to the tightener.

In an embodiment, each pivoting bracket comprises bushings integrated directly into each pivoting bracket.

### BRIEF DESCRIPTION OF THE DRAWINGS

FIG. 1 is a front perspective view of a knee brace in accordance with an illustrative embodiment of the present invention;

FIG. 2 is left side view of a knee brace in accordance with an illustrative embodiment of the present invention;

FIG. 3 is a right side view of a knee brace in accordance with an illustrative embodiment of the present invention;

FIG. 4 is a front perspective view of a knee brace with a disconnected tightening mechanism, in accordance with an illustrative embodiment of the present invention;

FIG. 5 is a left side view of a knee brace with a disconnected tightening mechanism, in accordance with an illustrative embodiment of the present invention; and

FIGS. 6A and 6B are respective front and front perspective views of a receptacle for a tightening mechanism for a knee brace, in accordance with an illustrative embodiment of the present invention;

FIG. 7 is a rear view of a knee brace in accordance with an illustrative embodiment of the present invention;

FIG. 8 is a right side view of upper and lower support members of a knee brace with integrated articulating bushings, in accordance with an illustrative embodiment of the present invention; and

FIG. 9 is a front perspective view of an integrated articulating bushing for a knee brace, in accordance with an illustrative embodiment of the present invention.

### DETAILED DESCRIPTION OF THE ILLUSTRATIVE EMBODIMENTS

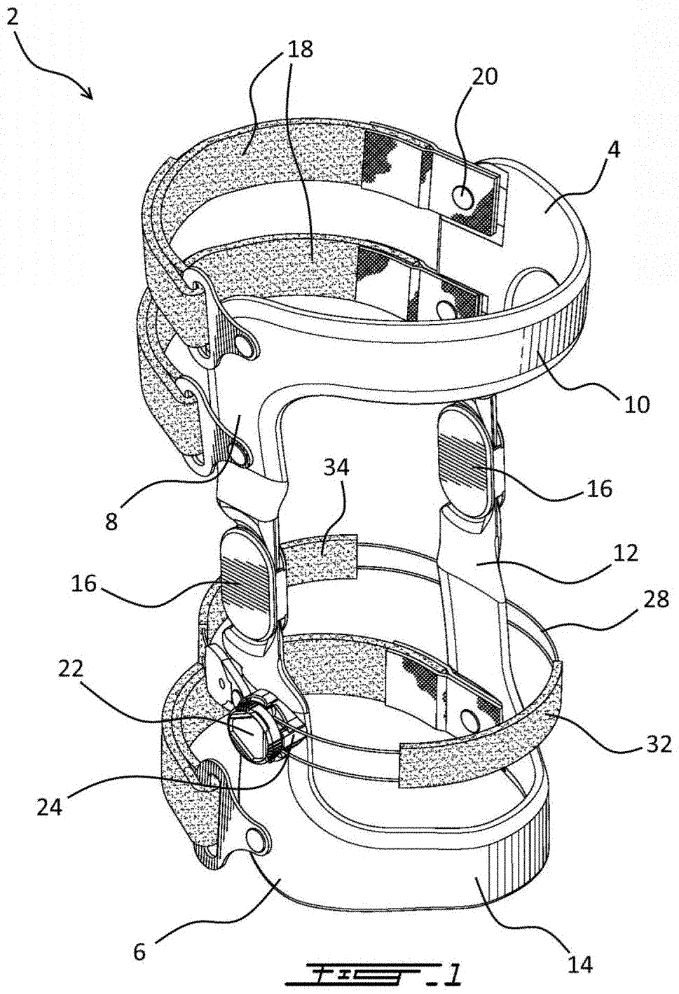

Referring to FIG. 1, there is shown a knee brace, generally referred to using the reference numeral 2. The knee brace 2 is designed to support and stabilize the knee of an individual or user for orthotic purposes, for example to recover from an injury. The knee brace 2 comprises an upper or femoral frame member 4 and a lower or tibial frame member 6. The upper frame member 4 comprises a pair of upper strut members 8 and an upper cross member 10 that presses against the user's upper leg. The lower frame member 6 comprises a pair of lower strut members 12 and a lower cross member 14 that presses against the user's lower leg. Upper frame member 4 and lower frame member 6 are pivotably linked by a pair of pivoting brackets 16. Each pivoting bracket 16 links a respective upper strut member 12 to a lower strut member 14, allowing the knee brace 2 to articulate and conform to the user's various leg movements. Knee brace 2 is attached to the user's leg by a plurality of retention straps 18, illustratively joined to the knee brace 2 by a plurality of pins 20. Knee brace 2 further comprises a tightening mechanism 22 that, when tightened around the user's leg, increases the rigidity of the knee brace 2 and provides circular compression of the knee brace 2 around the user's leg. Tightening mechanism 22 is attached to one of the lower frame members 6 of the knee brace 2 by being inserted into a receptacle 24 that is directly integrated into the knee brace 2, as will be discussed in further detail below.

Preferably, the upper frame member 4, lower frame member 6 and the pivoting brackets 16 are 3D-printed. The 3D-printing facilitates manufacturing of the brace 2 as it becomes easier to directly integrate the receptacle 24 onto the lower strut member 14 of the lower frame member 6. Compared with other manufacturing techniques, 3D-printing typically reduces costs, and results in a relatively resistant yet relatively light knee brace 2.

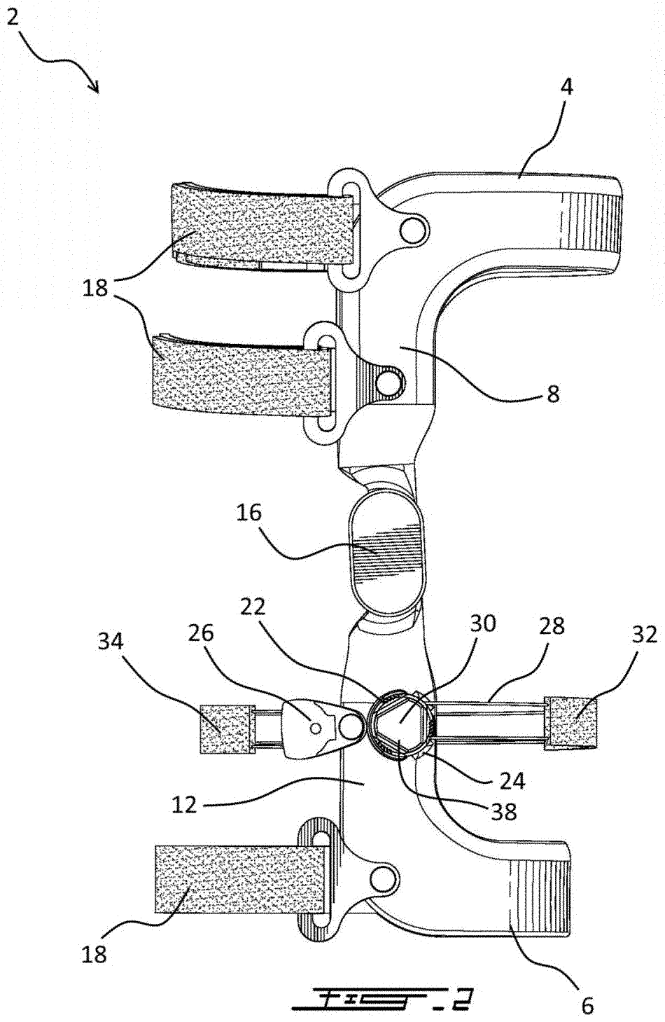

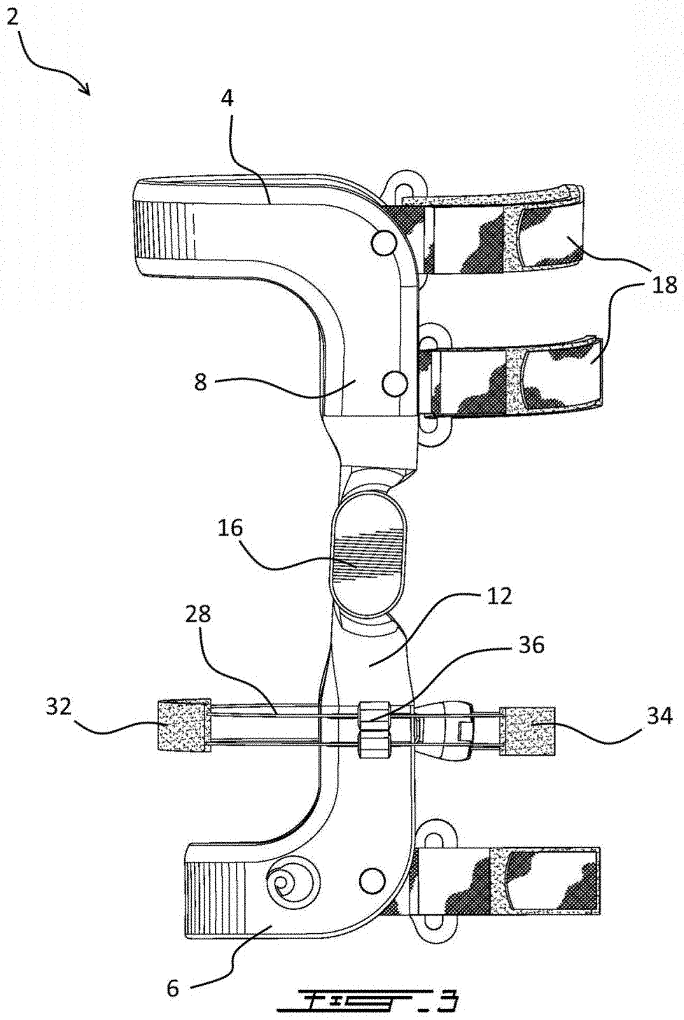

Referring now to FIGS. 2 and 3 in addition to FIG. 1, the tightening mechanism 22 comprises a connector 26 coupled to a tether 28 which is coupled to a tightener 30, illustratively a mechanical tensioner, mounted to a first one of the lower strut members 12. The tether 28, illustratively a pair of cables, passes through a front pad 32 and a rear pad 34 which press, respectively, against the front and the rear of the user's lower leg. The tether 28 further passes through a set of tether guides 36 on a second one of the opposing lower strut members 12. The connector 26 is engageable with the tightener 30, and the tether 28 is retractable by the tightener 30 to decrease the circumference of the tether 28, thus conforming the knee brace 2 to the user's leg and increasing the rigidity of the knee brace 2.

Still referring to FIGS. 2 and 3 in addition to FIG. 1, the tension in the tether 28 is controllable by the tightener 30, which is movable between a locked configuration and an unlocked configuration. In the locked configuration, the tightener 30 is configured to prevent extension of the tether 28 while nonetheless allowing the tether 28 to be tightened. In the unlocked configuration, the tightener 30 is configured to allow for extension of the tether 28, thus loosening the knee brace 2 from the user's leg. In an embodiment, the tightener 30 comprises a dial 38 that is rotatable to increase or decrease the tension of the tether 28. Further, the dial 38 is moveable in an axial direction to move the tightener 30 between the locked configuration and the unlocked configuration.

The exemplary tightening mechanism 22 shown in the figures embody the lacing system and lace lock technology manufactured by Boa Technology, Inc. For a more detailed discussion pertaining to the lacing system and lacing lock technology, please refer to U.S. Pat. Nos. 6,289,558 and 7,591,050, both entitled Footwear Lacing System. A person skilled in the art would appreciate that the Boa Lacing System is exemplary in nature only and that other tightening mechanisms may be used without departing from the spirit and scope of the present invention.

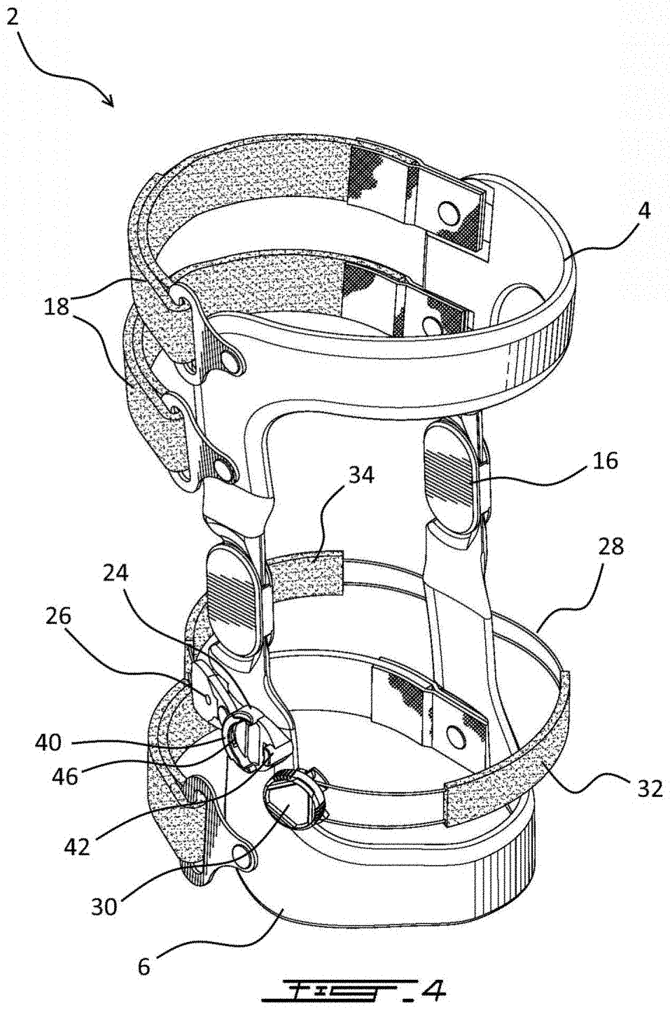

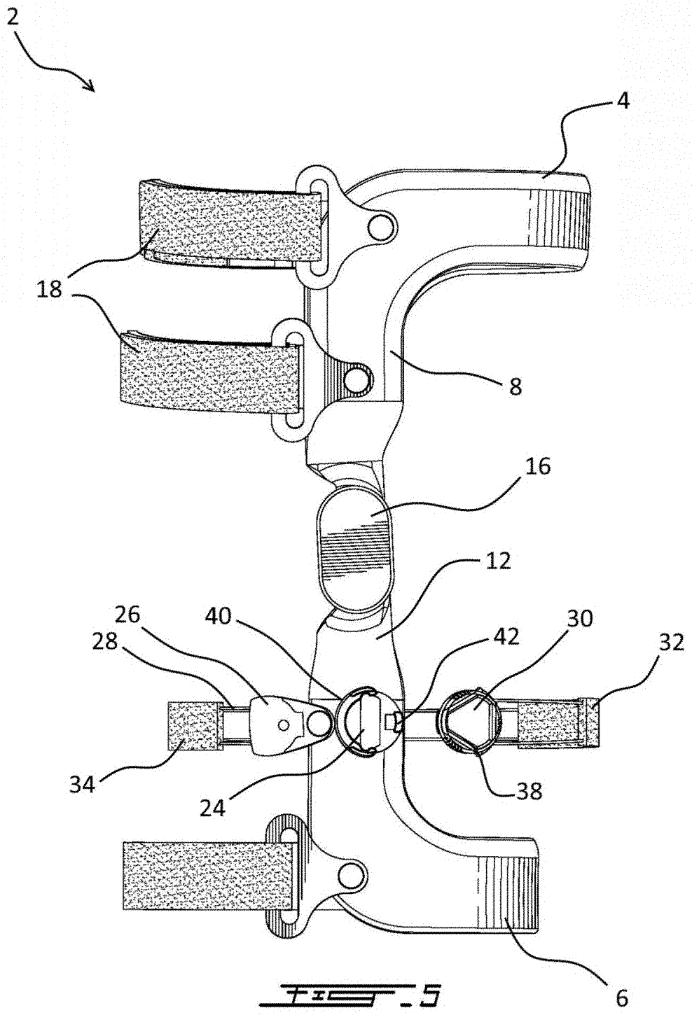

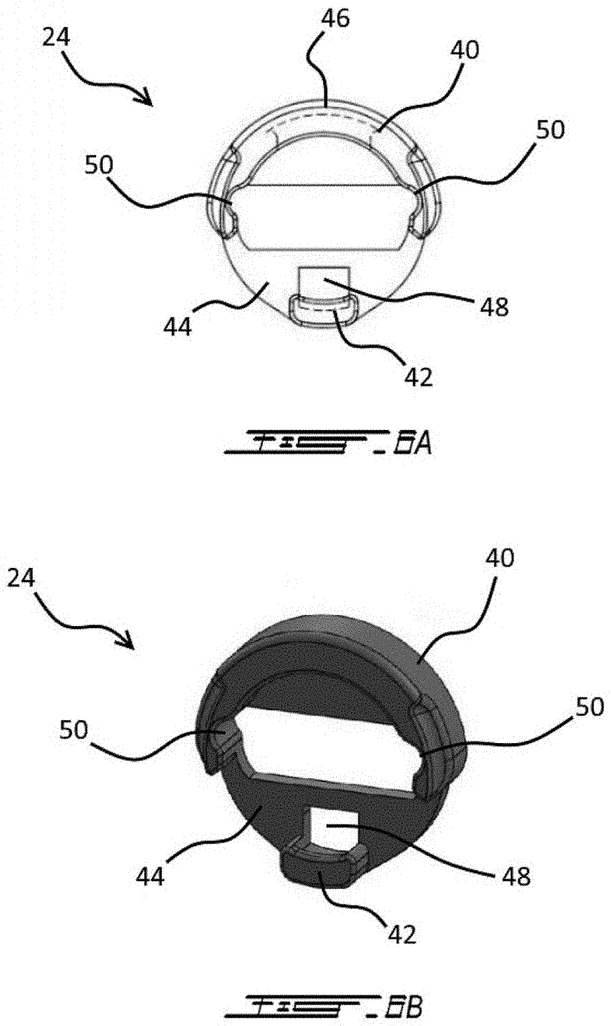

Referring now to FIGS. 4, 5, 6A and 6B, receptacle 24 comprises a rear receptacle wall 40 and a front receptacle wall 42 protruding from base 44 such that tightener 30 is insertable and retainable in receptacle 24. Receptacle 24 further comprises a slot 46 in which connector 26 is insertable such that it may connect to tightener 30. Additionally, slot 48 in the base 44 and cut-outs 50 in the rear receptacle wall engage with tightener 30 to facilitate with its engagement and retainment. As such, both connector 26 and tightener 30 are removable from receptacle 24, facilitating their replacement in case of damage or wear and tear. Advantageously, receptacle 24 is built directly into one of the lower strut members 12 of the knee brace 2 rather than being its own removable component. In an embodiment, receptacle 24 is 3D-printed directly onto lower strut member 12. This direct implementation allows connector 26 and tightener 30 to be directly inserted onto the knee brace 2 without the need for additional components which may break or wear out over time.

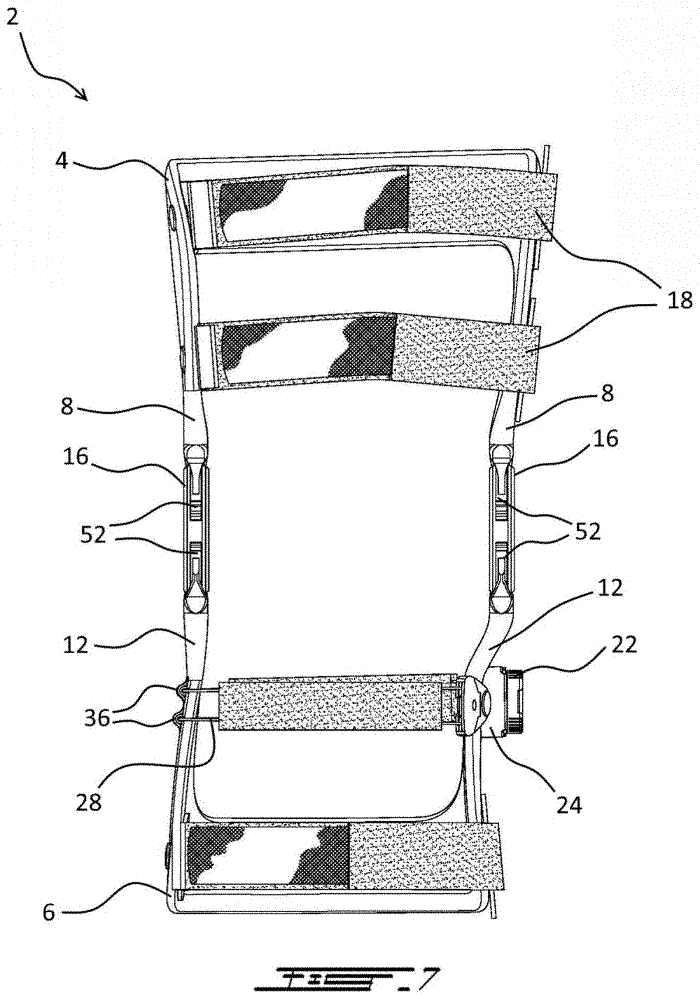

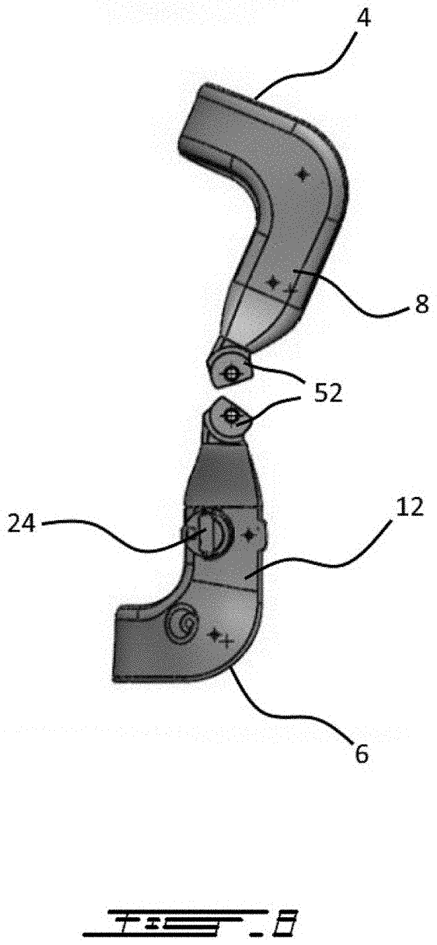

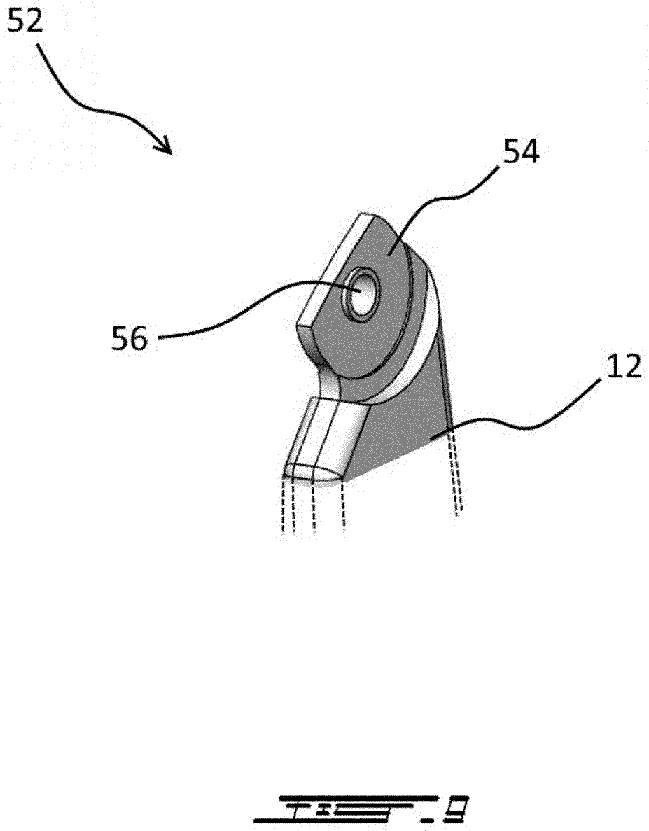

Referring now to FIGS. 7 to 9, in an embodiment, pivoting bracket 16 comprises a plurality of bushings 52 integrated directly into the pivoting bracket 16. Each bushing 52 is connected to a respective upper strut member 8 or lower strut member 12, allowing the knee brace 2 to articulate and conform to the user's various leg movements while offering the desired support. Each bushing 52 comprises a semi-circular disk 54 with a circular cut-out 56 to facilitate attachment to the pivoting bracket 16 by, for example, a pin (not shown). Advantageously, these directly integrated bushings 52 are more solid and more resistant to breaking than previously-implemented bushings that were inserted as separate components.

Referring back to FIG. 1 in addition to FIG. 4, in order to attach the knee brace 2 to a user's knee, the user would first grossly adjust the knee brace 2 to conform to the size and shape of the user's leg by tightening the retention straps 18. Then, the user would ensure that the tightening mechanism 22 is properly mounted to the knee brace 2 by verifying that the tightener 30 is inserted into the receptacle 24 and that the connector 26 is securely fastened to the tightener 30 by passing it through slot 46. Then, the user would use the tightening mechanism 22 to tension the tether 28 around the knee brace 2, thus increasing its rigidity and applying circular compression around the user's knee. Advantageously, the tightening mechanism 22 allows the user to increase the rigidity and circular compression around their knee rather than simply acting as a strap to retain the knee brace 2 around the user's leg.

The scope of the claims should not be limited by the preferred embodiments set forth in the examples, but should be given the broadest interpretation consistent with the description as a whole.

1. A knee brace (2) for supporting and stabilizing a knee, the knee brace (2) comprising:
an upper frame member (4);
a lower frame member (6);
a plurality of pivoting brackets (16), each said pivoting bracket (16) pivotably linking said upper frame member (4) to said lower frame member (6);
at least one retention strap (18), each said retention strap (18) holding the knee brace (2) on the knee;
a tightening mechanism (22), said tightening mechanism (22) controlling the rigidity of the knee brace (2) and the circular compression around the knee; and
a receptacle (24) for receiving said tightening mechanism (22), said receptacle (24) integrated directly into said lower frame member (6).

2. The knee brace of claim 1, wherein the upper frame member (4) is 3D-printed.

3. The knee brace of claim 1, wherein the lower frame member (6) is 3D-printed.

4. The knee brace of claim 1, wherein the pivoting brackets (16) are 3D-printed.

5. The knee brace of claim 1, wherein said upper frame member (4) comprises a pair of upper strut members (8) and an upper cross member (10), and said lower frame member (6) comprises a pair of lower strut members (12) and a lower cross member (14), wherein each said upper strut member (8) is joined to a corresponding lower strut member (12) at a respective said pivoting bracket (16), said upper cross member (10) applying pressure above the knee, and said lower cross member (14) applying pressure below the knee.

6. The knee brace of claim 5, wherein the upper frame member (4) is 3D-printed.

7. The knee brace of claim 5, wherein the lower frame member (6) is 3D-printed.

8. The knee brace of claim 5, wherein the pivoting brackets (16) are 3D-printed.

9. The knee brace of claim 7, wherein said receptacle (24) is 3D-printed onto a first one of said lower strut members (12) and comprises a base (44), a rear receptacle wall (40) and a front receptacle wall (42), said tightening mechanism (22) insertable between said rear receptacle wall (40) and said front receptacle wall (42).

10. The knee brace of claim 9, wherein the upper frame member (4) is 3D-printed.

11. The knee brace of claim 9, wherein the pivoting brackets (16) are 3D-printed.

12. The knee brace of claim 9, wherein said tightening mechanism (22) comprises:
a tightener (30) insertable into said receptacle (24), said tightener (30) comprising a rotatable and telescopable dial (38);
a connector (26) insertable into said tightener (30) to secure said tightening mechanism (22) into place;
a tether (28) tethered to said tightener (30) and said connector (26), said tether (28) circumferentially wrapping around the knee brace (2);
a front pad (32) and a rear pad (34), each said pad applying pressure below the front and the rear of the knee respectively, said tether (28) passing through said front pad (32) and said rear pad (34); and
tether guides (36) attached to a second one of said lower strut members, said tether guides retaining and guiding said tether (28);
wherein said tightening mechanism (22) is tightenable and loosenable by rotating said dial (38) on said tightener (30); and
wherein said tightening mechanism (22) is movable between a locked position and an unlocked position by axially adjusting said dial (38) on said tightener (30).

13. The knee brace of claim 12, wherein the upper frame member (4) is 3D-printed.

14. The knee brace of claim 12, wherein the pivoting brackets (16) are 3D-printed.

15. The knee brace of claim 12, wherein said receptacle (24) further comprises a slot (48) in said base (44) and a plurality of cut-outs (50) in said rear receptacle wall (42), said slot (48) and plurality of cut-outs (50) engageable with said tightener (28), said receptacle (24) further comprising a slot (46) in said rear receptacle wall (42) such that said connector (26) is insertable through said slot (46) in said rear receptacle wall (42) to connect to said tightener (28).

16. The knee brace of claim 1, wherein each said pivoting bracket (16) comprises a bushing (52) integrated directly into each said pivoting bracket (16).

17. The knee brace of claim 4, wherein each said pivoting bracket (16) comprises a bushing (52) integrated directly into each said pivoting bracket (16).

18. The knee brace of claim 9, wherein each said pivoting bracket (16) comprises a bushing (52) integrated directly into each said pivoting bracket (16).

19. The knee brace of claim 12, wherein each said pivoting bracket (16) comprises a bushing (52) integrated directly into each said pivoting bracket (16).

20. The knee brace of claim 15, wherein each said pivoting bracket (16) comprises a bushing (52) integrated directly into each said pivoting bracket (16).
